(19) United States
(12) Patent Application Publication
(10) Pub. No.: US 2025/0058906 A1
(43) Pub. Date: Feb. 20, 2025

(54) ADAPTIVE DETECTION AND DIFFUSION OF CARBON DIOXIDE USING DRONES

(71) Applicant: International Business Machines Corporation, Armonk, NY (US)

(72) Inventors: Sarbajit K. Rakshit, Kolkata (IN); Manikandan Padmanaban, Chennai (IN); Jagabondhu Hazra, Bangalore (IN)

(73) Assignee: International Business Machines Corporation, Armonk, NY (US)

(21) Appl. No.: 18/235,010

(22) Filed: Aug. 17, 2023

(51) Int. Cl. B64U 20/80; B64U 101/70; G01N 33/00

(52) U.S. Cl. CPC B64U 20/80; G01N 33/004; B64U 2101/70

(57) ABSTRACT

An embodiment includes sending a first drone, including a carbon dioxide sensor, into an area. The embodiment includes detecting a level of carbon dioxide in the area and generating a map including a position and level of carbon dioxide. The embodiment includes calculating a carbon dioxide hotspot above a threshold. The embodiment includes creating a plan to diffuse the carbon dioxide hotspot in a time limit by calculating an amount of a drones to send to the area based on a needed amount of humidity and light to diffuse the carbon dioxide hotspot in the time limit. The amount of humidity and light are calculated using an optimizer module. The embodiment includes sending the calculated amount of drones to the area of the carbon dioxide hotspot. The embodiment includes diffusing the carbon dioxide hotspot, sampling the area, and updating the map with new levels of carbon dioxide.

### BACKGROUND

The present invention relates generally to artificial photosynthesis. More particularly, the present invention relates to a method, system, and computer program for piloting autonomous drones with artificial photosynthesis modules to reduce carbon dioxide gas.

Artificial photosynthesis is a chemical process that replicates natural photosynthesis to reduce carbon dioxide gas. Artificial photosynthesis can be facilitated using polymer membranes. The membranes function as an artificial leaf to utilize light and humidity to diffuse carbon dioxide gas into hydrogen gas and other hydrocarbon fuels.

Excess carbon dioxide gas can present a health risk to people. While entering a mining passage, inspecting an abandoned building or well accumulated carbon dioxide can present a health risk to both the rescuers and workers. Carbon dioxide is heavy and so it accumulates in low places such as mines, wells, basements, etc. Drones can be used to fly into these places before humans enter to detect the levels of carbon dioxide in the space. If carbon dioxide is detected above a threshold, additional drones with artificial photosynthesis modules can be sent into the places to convert and diffuse the carbon dioxide.

### SUMMARY

The illustrative embodiments provide for adaptive detection of carbon dioxide. An embodiment includes sending a first drone into an area. The first drone includes a carbon dioxide sensor. The embodiment also includes detecting a level of carbon dioxide gas in the area. The embodiment also includes generating a map where the map includes a position and the level of carbon dioxide. The embodiment also includes calculating a carbon dioxide hotspot where the level of carbon dioxide is above a threshold. The embodiment also includes creating a plan, using a planner module, for diffusing the carbon dioxide hotspot in a time limit. Creating the plan includes calculating an amount of a drones to send to the area based on a needed amount of humidity and a needed amount of light to diffuse the carbon dioxide hotspot in the time limit. The needed amount of humidity and the needed amount of light are calculated using an optimizer module. The time limit is provided by a work order. The embodiment also includes sending the calculated amount of drones to the area of the carbon dioxide hotspot. The embodiment also includes diffusing the carbon dioxide hotspot, using the amount of drones. The embodiment also includes sampling the area, using the first drone, and updating the map with new levels of carbon dioxide. Other embodiments of this aspect include corresponding computer systems, apparatus, and computer programs recorded on one or more computer storage devices, each configured to perform the actions of the embodiment.

An embodiment includes a computer usable program product. The computer usable program product includes a computer-readable storage medium, and program instructions stored on the storage medium.

An embodiment includes a computer system. The computer system includes a processor, a computer-readable memory, and a computer-readable storage medium, and program instructions stored on the storage medium for execution by the processor via the memory.

### BRIEF DESCRIPTION OF THE DRAWINGS

The novel features believed characteristic of the invention are set forth in the appended claims. The invention itself, however, as well as a preferred mode of use, further objectives, and advantages thereof, will best be understood by reference to the following detailed description of the illustrative embodiments when read in conjunction with the accompanying drawings, wherein:

FIG. 1 depicts a block diagram of a computing environment in accordance with an illustrative embodiment;

FIG. 2 depicts a block diagram of an example artificial photosynthesis system in accordance with an illustrative embodiment; and

FIG. 3 depicts a block diagram of an example system architecture in accordance with an illustrative embodiment.

### DETAILED DESCRIPTION

While entering inside any mining passage, or inspecting any abandoned building, well, rescuing people from any building, or passage, tunnel, discovery of cave, etc., a health concern is accumulation of carbon dioxide. This creates a problem for human mobility, and so, the human workers need to wear masks, and carry oxygen cylinders, etc. Carbon dioxide is heavy, and it accumulates in low areas such as mines, caves, basements, etc. Carbon dioxide needs to be removed from the areas to allow human mobility for mining, rescues, exploration, and other activities.

Similarly, black damp is the mining term for a mixture of carbon dioxide and other unbreathable gases that can build-up in mines causing poisoning, asphyxiation, and ultimately death if left untreated. Exhaust fans are used to pump out carbon dioxide gas from underground mines. However, these fans do not completely avoid or remove black damps. There is a growing number of fatalities among miners caused by toxic gases.

Carbon dioxide released in underground mines cannot easily be detected by human senses because the gas is not visible and does not have a smell. There is a need to locate and diffuse carbon dioxide gases in enclosed areas such as mines, wells, basements, abandoned buildings, etc. Carbon dioxide could be proactively diffused using autonomous piloting devices containing artificial photosynthesis modules.

Therefore, if human mobility needs to be performed through any passage, and there is a possibility to have accumulated carbon dioxide in the surrounding passage, then there is a need for a method to remove carbon dioxide from the area to allow people to work safely in the area. Artificial Photosynthesis may be used to recover fuel from the accumulated carbon dioxide in the above-mentioned areas and therefore create a safer place for people to work.

Artificial photosynthesis is a chemical process that replicates natural photosynthesis and may reduce carbon dioxide (CO₂) gas, increase fuel security, and provide a sustainable global economy. Artificial photosynthesis may be done using artificial leaves for the efficient conversion of solar energy into hydrogen (H₂) gas and other fuels. Artificial “leaves” may be made of, by non-limiting example, synthetic polymers with ionic properties such as sulfonated tetrafluoroethylene. Artificial photosynthesis can also be performed using photoelectrochemical cells (PEC) as a catalyst to produce hydrocarbon fuels using carbon dioxide, light, and humidity to create hydrocarbon fuels.

The present disclosure addresses the need to reduce excess CO₂ gas especially in small workspaces to make the areas safe for human movement. Artificial photosynthesis can be used to decrease the CO₂ gas. However, there is need to get the artificial photosynthesis modules into the areas with excess CO₂ without human contact. The present disclosure proposes a solution to the deficiencies described above by providing a process (as well as a system, method, machine-readable medium, etc.) that creates the conducive environment for artificial photosynthesis reaction including detecting CO₂ concentration, determining the optimal proportion of water and irradiance for a forecasted time limit by considering the capacities of artificial photosynthesis units, photon generators, and humidity controllers.

The illustrative embodiments provide for adaptive detection and diffusion of carbon dioxide using autonomous drones. A drone as referred to herein is unmanned aerial vehicle (UAV). An artificial photosynthesis module as referred to herein is a device capable of using light, humidity, and CO₂ gas to produce hydrogen gas or other hydrocarbon fuels. An excess of CO₂ gas may be above a threshold of 5000 parts per million (ppm). A threshold may also be below 40,000 ppm. The American Conference of Governmental Industrial Hygienists (ACGIH) recommends an 8-hour Threshold Limit Value (TLV) of 5,000 ppm and a Ceiling exposure limit (not to be exceeded) of 30,000 ppm for a 10-minute period. A value of 40,000 is considered immediately dangerous to life and health (IDLH value). Embodiments disclosed herein describe a system of drones to diffuse excess CO₂; however, use of this example is not intended to be limiting but is instead used for descriptive purposes only. Instead, the artificial photosynthesis modules and related computer implements methods can be used with other devices that are able to access an area before people enter to diffuse CO₂ gas. A geo-tagged map as referred to herein is a map with geographical metadata added to the carbon dioxide levels. For example, a map will be generated showing various levels of carbon dioxide in an area sampled by a drone and the map will also give the geographical location of the area.

Illustrative embodiments include sending a first drone into an area, the first drone including a carbon dioxide (CO₂) sensor. In some implementations, the first drone may be autonomous following a known flight pattern. In other implementations, the first drone may be directed by a user to an area suspected to have carbon dioxide levels over a threshold. The first drone may detect a level of carbon dioxide in the area. The system may generate a map including a position and the levels of the carbon dioxide. The map may be a geo-tagged map. A geo-tagged map as referred to herein is a map with geographical metadata added to the carbon dioxide levels.

Illustrative embodiments include calculating a carbon dioxide hotspot. A hotspot as referred to herein is a portion of the sampled area where the carbon dioxide levels are above a threshold. A threshold as referred to herein is an amount of carbon dioxide that is harmful or deadly to a human.

Illustrative embodiments also include creating a plan for diffusing the carbon dioxide hotspot in a time limit. The time limit may be provided by a work order. A time limit may include a time before people need to be in the area such as for mining, excavating, or other similar activities. Creating the plan includes calculating an amount of a drones to send to the area based on a needed amount of humidity and a needed amount of light to diffuse the carbon dioxide hotspot in the time limit. Calculating the amount of drones needed includes using a calculation taking into account the level of carbon dioxide in the area, the absorption of carbon dioxide at the hotspot, the number of artificial photosynthesis units, photo generators, and humidity units. The needed amount of humidity and the needed amount of light may be calculated using an optimizer module.

Illustrative embodiments respond to creating a plan for diffusing the carbon dioxide by sending the calculated amount of drones to the area of the carbon dioxide hotspot. The calculated amount of drones may include a swarm of drones. A swarm of drones as referred to herein includes a group of drones operating together autonomously to accomplish a specific task. The drones may diffuse the carbon dioxide hotspot using the artificial photosynthesis module. The first drone may then sample the area again and update the map with the new level of carbon dioxide.

Illustrative embodiments may also include determining the new level of carbon dioxide is above the threshold and may create a new plan to resend or send additional drones to diffuse the carbon dioxide.

For the sake of clarity of the description, and without implying any limitation thereto, the illustrative embodiments are described using some example configurations. From this disclosure, those of ordinary skill in the art will be able to conceive many alterations, adaptations, and modifications of a described configuration for achieving a described purpose, and the same are contemplated within the scope of the illustrative embodiments.

Furthermore, simplified diagrams of the data processing environments are used in the figures and the illustrative embodiments. In an actual computing environment, additional structures or components that are not shown or described herein, or structures or components different from those shown but for a similar function as described herein may be present without departing the scope of the illustrative embodiments.

Furthermore, the illustrative embodiments are described with respect to specific actual or hypothetical components only as examples. Any specific manifestations of these and other similar artifacts are not intended to be limiting to the invention. Any suitable manifestation of these and other similar artifacts can be selected within the scope of the illustrative embodiments.

The examples in this disclosure are used only for the clarity of the description and are not limiting to the illustrative embodiments. Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Furthermore, the illustrative embodiments may be implemented with respect to any type of data, data source, or access to a data source over a data network. Any type of data storage device may provide the data to an embodiment of the invention, either locally at a data processing system or over a data network, within the scope of the invention. Where an embodiment is described using a mobile device, any type of data storage device suitable for use with the mobile device may provide the data to such embodiment, either locally at the mobile device or over a data network, within the scope of the illustrative embodiments.

The illustrative embodiments are described using specific code, computer readable storage media, high-level features, designs, architectures, protocols, layouts, schematics, and tools only as examples and are not limiting to the illustrative embodiments. Furthermore, the illustrative embodiments are described in some instances using particular software, tools, and data processing environments only as an example for the clarity of the description. The illustrative embodiments may be used in conjunction with other comparable or similarly purposed structures, systems, applications, or architectures. For example, other comparable mobile devices, structures, systems, applications, or architectures, therefore, may be used in conjunction with such embodiment of the invention within the scope of the invention. An illustrative embodiment may be implemented in hardware, software, or a combination thereof.

The examples in this disclosure are used only for the clarity of the description and are not limiting to the illustrative embodiments. Additional data, operations, actions, tasks, activities, and manipulations will be conceivable from this disclosure and the same are contemplated within the scope of the illustrative embodiments.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing.

Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

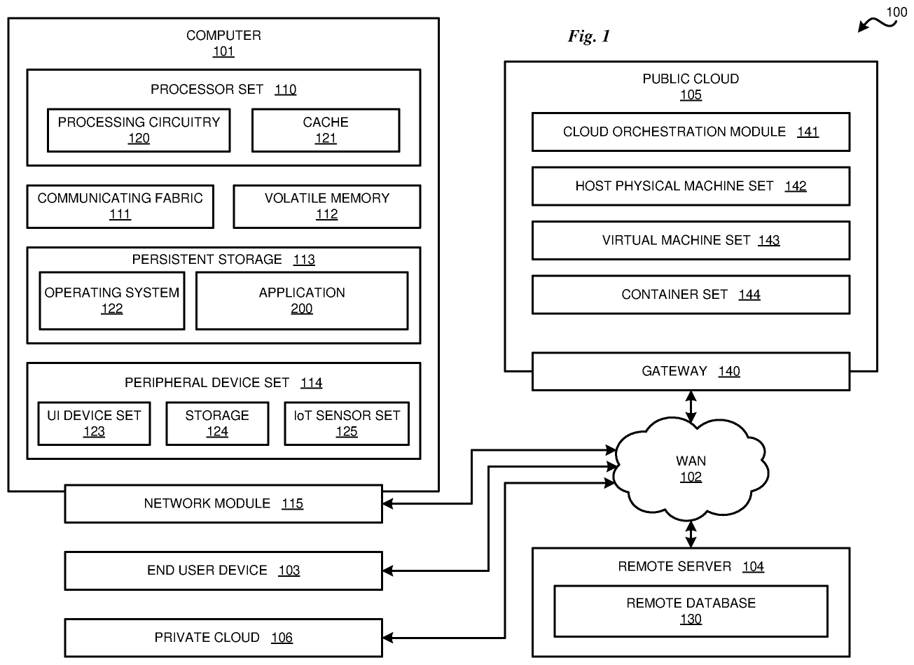

With reference to FIG. 1, this figure depicts a block diagram of a computing environment 100. Computing environment 100 contains an example of an environment for the execution of at least some of the computer code involved in performing the inventive methods, such as an application 200 that calculates a level of carbon dioxide in an area and calculates the required number of artificial photosynthesis modules required to diffuse the level of carbon dioxide. In addition to application 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and application 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in application 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in application 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 012 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Measured service: cloud systems automatically control and optimize resource use by leveraging a metering capability at some level of abstraction appropriate to the type of service (e.g., storage, processing, bandwidth, and active user accounts). Resource usage can be monitored, controlled, reported, and invoiced, providing transparency for both the provider and consumer of the utilized service.

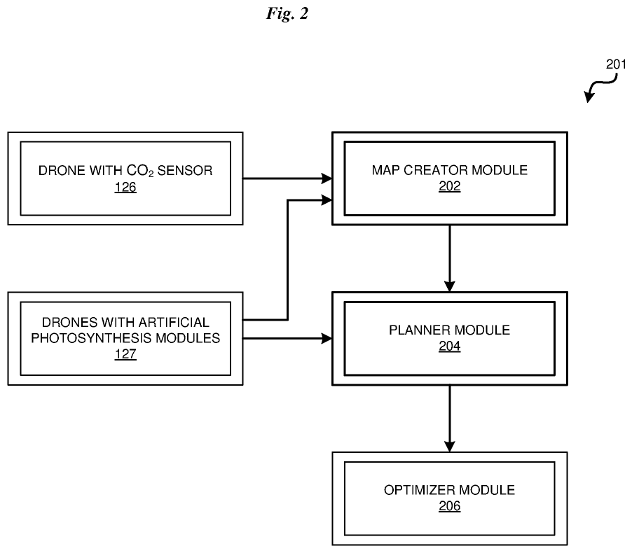

With reference to FIG. 2, this figure depicts a block diagram of an example artificial photosynthesis system 201 in accordance with an illustrative embodiment. In the illustrated embodiment, the example artificial photosynthesis system 201 includes the application 200 of FIG. 1.

In the illustrated embodiment, the artificial photosynthesis system 201 samples and processes the data from an area suspected of having high levels of CO₂. The artificial photosynthesis system includes modules in the application 200 as well as items in the peripheral device set 114 including a first drone 126 and a fleet of drones 127. Each drone in the fleet of drones may include an artificial photosynthesis unit (APU), a photon generator, and a humidity controller unit. A drone 126 is sent to an area to a level of sample CO₂. In some implementations, the drones may be directed by a user with a camera in the drone to aid in positioning. In other implementations, the drones may be autonomous and sent to a known location using the global positioning system (GPS). The drone 126 communicates with the map creator module 202. The drone may send the information over a network such as the internet. The map creator module 202 creates a geo-tagged map where the geo-tagged map gives both the geographical location and the CO₂ levels at the locations. If CO₂ hotspots are detected the information from the map is communicated to the planner module 204.

In the illustrated embodiment, the planner module 204 creates a plan for diffusing the carbon dioxide hotspot in a time limit where creating the plan includes calculating an amount of a drones to send to the area based on a needed amount of humidity and a needed amount of light to diffuse the carbon dioxide hotspot in the time limit. The calculations include the number of artificial photosynthesis units (APU), photon generators, and humidity controllers are needed.

The plan creates the plan using the following calculations:

$$J = Min ( \frac{{Cint}_{1}^{t}}{{C}_{1}} + \frac{{Cint}_{2}^{t}}{{C}_{2}} + \frac{{Cint}_{3}^{t}}{{C}_{3}} + … + \frac{{Cint}_{n}^{t}}{{C}_{n}} )$$

Where, Cint₁^{t} is the CO₂ level at a hotspot 1 at time t; C₁ is the CO₂: C₁=f (x1.APU, x2.PG, x3.HC); APU: Artificial Photosynthesis Unit; PG: Photon generator; HC: Humidity controller; x1,x2,x3 # of selected units.

Carbon hotspots are portions of the area where the carbon dioxide levels are above a threshold. In various implementations, the threshold may be above 5000 parts per million (ppm). In other implementations, the threshold may be above 5000 ppm and below 40,000 ppm. In some implementations, the threshold of carbon dioxide may be set by a governing agency.

The plan created by the planner module 204 is then sent to the optimizer module 206. The optimizer module 206 calculates the needed amount of humidity and the needed amount of light. The optimizer module also takes into account the type of catalyst to be used by the artificial photosynthesis units. The artificial photosynthesis units include a catalyst to act as an artificial leaf to facilitate the chemical reaction diffusing carbon dioxide into hydrocarbon fuel. The catalyst may be a synthetic polymer with ionic properties. In various implementations, the catalyst may include a photocatalyst, photochemical, bio electrochemical, etc. The optimizer module includes a knowledge database of catalyst reaction profiles and sensitivities.

The control of humidity and light must be very precise and should allow for small changes in each step while taking the whole prediction horizon into account. Such as, by non-limiting example, the humidity should be chosen for next “K” steps considering that sudden changes in the humidity are unlikely to occur whether it is an increase or a decrease. The calculation used by the optimizer module may include:

$$J = {\sum }_{i = 1}^{N} {{w}_{{H}_{i}} ( {H}_{i}^{r} - {H}_{i}^{m} )}^{2} + {\sum }_{i = 1}^{N} {{w}_{{L}_{i}} ( {L}_{i}^{r} - L )}^{2} + {\sum }_{i = 1}^{N} {{w}_{{uH}_{i}} ( Δ u )}^{2} + {\sum }_{i = 1}^{N} {{w}_{{vL}_{i}} ( Δ v )}^{2} subject to 0 \leq {H}_{i}^{m} \leq {H}^{max} ;$$

$$0 \leq {L}_{i}^{m} \leq {L}^{max}$$

Where J—cost function over the receding horizon;
Hᵢ^{r}—required Humidity for the instant ‘i’ from the knowledge base;
Hᵢ^{m}—measured humidity for the instant ‘i’;
Lᵢ^{r}—required light intensity for the instant ‘i’ from the knowledge base;
Lᵢ^{m}—measured light intensity for the instant ‘i’;
u,v—humidity and light intensity controller variable respectively;
w_{H}_{ᵢ},w_{L}_{ᵢ}—weighting coefficient for humidity and light respectively;
w_{uH}_{ᵢ},w_{uL}_{ᵢ}—penalizing coefficient for big changes in humidity and light intensity controller respectively; and
H^{max}, L^{max}—Maximum limit for humidity and light intensity level respectively;

Once the plan has been optimized, the system sends the calculated amount of drones to the area of the carbon dioxide hotspot. The calculated amount of drones may be a fleet of drones or a swarm of drones where a swarm of drones is a group of drones programmed to work collaboratively on a common goal. The amount of drones diffuses the carbon dioxide hotspot in the time limit provided in the plan. The time limit may be provided by a user inputting a work order into the system. The time limit may take into account when people need to enter the area. The optimizer module may decrease a time from the planner module by increasing an amount of catalyst to make the artificial photosynthesis reaction occur more quickly.

After the calculated amount of drones has diffused the carbon dioxide hotspots, the first drone 126 may return to the area to sample the air in the area and create a new map using the map creator module 202. If the carbon dioxide hotspots are diffused, meaning that the carbon dioxide levels are below a threshold, the area may be approved for people to enter the area. If the carbon dioxide hotspots are not diffused, the system may determine the new level of carbon dioxide is above a threshold and may create a new plan using the planner module 204 and the optimized module 206. The new plan may include diffusing the new level of carbon dioxide using a calculated a calculated number of drones. The new plan may include creating an updated carbon dioxide map showing wherein the carbon dioxide levels are below the threshold of carbon dioxide.

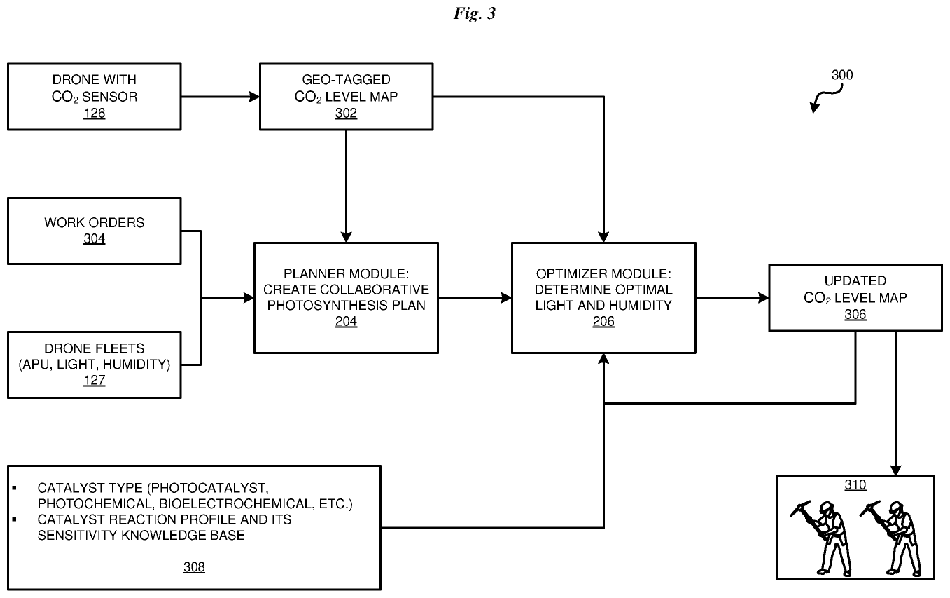

With reference to FIG. 3, this figure depicts a block diagram of an exemplary system architecture 300 in accordance with an illustrative embodiment.

In the illustrated embodiment, the first drone 126 is sent to an area. The first drone 126 has a CO₂ sensor. Using the sensor, the first drone 126 detects a level of carbon dioxide in an area. The first drone sends the information from the sensor to the map module to create a geo-tagged CO₂ level map. The geo-tagged map shows the position of the carbon dioxide and the levels of the CO₂. The geo-tagged map is sent to the planner module 204. In the planner module 204, a plan is created for diffusing the carbon dioxide hotspot in a time limit. The time limit may be pre-loaded into the planner module using work order 304 supplied by a user. In other implementations, the time limit may be determined by the planner module based on the levels of carbon dioxide and the materials available. In still other implementations, such as a rescue, the time may be calculated and changed to facilitate the situation. The plan determines the needed amount of light and needed amount of humidity needed to diffuse the carbon dioxide in a given time. Information about the drones having artificial photosynthesis units is used by the planner module in the calculations. The calculations may be performed using the formulas previously described.

The plan is sent to optimizer module 206. The optimizer module determines the optimal light and humidity needed to fulfill the plan. The optimizer module included information about catalysts 308 that can be used to increase the speed of the reaction or decrease the needed amount of light or humidity for the reaction in a given time. In various implementations, light might be provided by photon generators. In other implementations, other light sources may be used in the artificial photosynthesis reaction.

The amount of calculated drones is sent as a drone fleet 127 to the area to diffuse the carbon dioxide hotspots based on the optimized plan. After the carbon dioxide hotspots have been diffused, the area is sampled to determine new levels of carbon dioxide in the area. The area may be sampled by the first drone in various embodiments. In other embodiments, the drone fleet may include a sensor to determine the levels of carbon dioxide after the plan has been completed to diffuse the carbon dioxide. The map 306 is updated based on the sampling of the area. The system may also create an AR interface 310 on the map to show which areas of the map have reduced carbon dioxide.

In various embodiments, the system may also allow for the recovery of fuel from the artificial photosynthesis reaction. The fuel may be, by non-limiting example, hydrogen gas or other hydrocarbon fuels. The fuel that is created may be collected and stored by the drones in the drone fleet.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “illustrative” is used herein to mean “serving as an example, instance or illustration.” Any embodiment or design described herein as “illustrative” is not necessarily to be construed as preferred or advantageous over other embodiments or designs. The terms “at least one” and “one or more” are understood to include any integer number greater than or equal to one, i.e., one, two, three, four, etc. The terms “a plurality” are understood to include any integer number greater than or equal to two, i.e., two, three, four, five, etc. The term “connection” can include an indirect “connection” and a direct “connection.”

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terms “about,” “substantially,” “approximately,” and variations thereof, are intended to include the degree of error associated with measurement of the particular quantity based upon the equipment available at the time of filing the application. For example, “about” can include a range of +8% or 5%, or 2% of a given value.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

Thus, a computer implemented method, system or apparatus, and computer program product are provided in the illustrative embodiments for managing participation in online communities and other related features, functions, or operations. Where an embodiment or a portion thereof is described with respect to a type of device, the computer implemented method, system or apparatus, the computer program product, or a portion thereof, are adapted or configured for use with a suitable and comparable manifestation of that type of device.

Where an embodiment is described as implemented in an application, the delivery of the application in a Software as a Service (SaaS) model is contemplated within the scope of the illustrative embodiments. In a SaaS model, the capability of the application implementing an embodiment is provided to a user by executing the application in a cloud infrastructure. The user can access the application using a variety of client devices through a thin client interface such as a web browser (e.g., web-based e-mail), or other light-weight client-applications. The user does not manage or control the underlying cloud infrastructure including the network, servers, operating systems, or the storage of the cloud infrastructure. In some cases, the user may not even manage or control the capabilities of the SaaS application. In some other cases, the SaaS implementation of the application may permit a possible exception of limited user-specific application configuration settings.

Embodiments of the present invention may also be delivered as part of a service engagement with a client corporation, nonprofit organization, government entity, internal organizational structure, or the like. Aspects of these embodiments may include configuring a computer system to perform, and deploying software, hardware, and web services that implement, some or all of the methods described herein. Aspects of these embodiments may also include analyzing the client's operations, creating recommendations responsive to the analysis, building systems that implement portions of the recommendations, integrating the systems into existing processes and infrastructure, metering use of the systems, allocating expenses to users of the systems, and billing for use of the systems. Although the above embodiments of present invention each have been described by stating their individual advantages, respectively, present invention is not limited to a particular combination thereof. To the contrary, such embodiments may also be combined in any way and number according to the intended deployment of present invention without losing their beneficial effects.

What is claimed is:

1. A computer-implemented method comprising:
sending a first drone into an area, the first drone comprising a carbon dioxide sensor;
detecting a level of carbon dioxide in the area;
generating a map wherein the map includes a position and the level of carbon dioxide;
calculating a carbon dioxide hotspot where the level of carbon dioxide is above a threshold;
creating a plan, using a planner module, to diffuse the carbon dioxide hotspot in a time limit wherein creating the plan comprises calculating an amount of a drones to send based on a needed amount of humidity and a needed amount of light to diffuse the carbon dioxide hotspot in the time limit, wherein the needed amount of humidity and the needed amount of light is calculated using an optimizer module, wherein the time limit is provided by a work order;
sending the calculated amount of drones to the area of the carbon dioxide hotspot;
diffusing the carbon dioxide hotspot, using the calculated amount of drones;
sampling the area, using the first drone, and updating the map with new levels of carbon dioxide.

2. The computer-implemented method of claim 1, wherein each drone of the amount of drones comprises an artificial photosynthesis module.

3. The computer-implemented method of claim 2, wherein the artificial photosynthesis module comprises a humidity unit, a photon generator, and a catalyst.

4. The computer-implemented method of claim 3, wherein the catalyst is a synthetic polymer with ionic properties.

5. The computer-implemented method of claim 1, further comprising:
calculating the new levels of carbon dioxide are above the threshold;
creating a new plan; and
diffusing the carbon dioxide using a calculated number of drones.

6. The computer-implemented method of claim 1, wherein the needed amount of light is provided by a photon generator on a drone.

7. The computer-implemented method of claim 1, wherein the threshold of carbon dioxide is above 5,000 ppm.

8. The computer-implemented method of claim 1, wherein the threshold of carbon dioxide is below 40,000 ppm.

9. The computer-implemented method of claim 1, further comprising creating an updated carbon dioxide map showing where the level of carbon dioxide is below the threshold of carbon dioxide.

10. The computer-implemented method of claim 1, wherein the threshold is set by a governing agency.

11. A computer program product comprising one or more computer readable storage media, and program instructions collectively stored on the one or more computer readable storage media, the program instructions executable by a processor to cause the processor to perform operations comprising:
sending a first drone into an area, the first drone comprising a carbon dioxide sensor;
detecting a level of carbon dioxide in the area;
generating a map wherein the map includes a position and the level of carbon dioxide;
calculating a carbon dioxide hotspot where the level of carbon dioxide is above a threshold;
creating a plan, using a planner module, to diffuse the carbon dioxide hotspot in a time limit wherein creating the plan comprises calculating an amount of a drones to send based on a needed amount of humidity and a needed amount of light to diffuse the carbon dioxide hotspot in the time limit, wherein the needed amount of humidity and the needed amount of light is calculated using an optimizer module, wherein the time limit is provided by a work order;
sending the calculated amount of drones to the area of the carbon dioxide hotspot;
diffusing the carbon dioxide hotspot, using the calculated amount of drones;
sampling the area, using the first drone, and updating the map with new levels of carbon dioxide.

12. The computer program product of claim 11, wherein the stored program instructions are stored in a computer readable storage device in a data processing system, and wherein the stored program instructions are transferred over a network from a remote data processing system.

13. The computer program product of claim 11, wherein each drone of the amount of drones comprises an artificial photosynthesis module.

14. The computer program product claim 13, wherein the artificial photosynthesis module comprises a humidity unit, a photon generator, and a catalyst.

15. The computer program product claim 11, further comprising:
calculating the new levels of carbon dioxide are above the threshold;
creating a new plan; and
diffusing the carbon dioxide using a calculated number of drones.

16. The computer program product claim 15, further comprising creating an updated carbon dioxide map showing where the level carbon dioxide is below the threshold of carbon dioxide.

17. A computer system comprising a processor and one or more computer readable storage media, and program instructions collectively stored on the one or more computer readable storage media, the program instructions executable by the processor to cause the processor to perform operations comprising:
sending a first drone into an area, the first drone comprising a carbon dioxide sensor;
detecting a level of carbon dioxide in the area;
generating a map wherein the map includes a position and the level of carbon dioxide;
calculating a carbon dioxide hotspot where the level of carbon dioxide is above a threshold;
creating a plan, using a planner module, to diffuse the carbon dioxide hotspot in a time limit wherein creating the plan comprises calculating an amount of a drones to send based on a needed amount of humidity and a needed amount of light to diffuse the carbon dioxide hotspot in the time limit, wherein the needed amount of humidity and the needed amount of light is calculated using an optimizer module, wherein the time limit is provided by a work order;
sending the calculated amount of drones to the area of the carbon dioxide hotspot;
diffusing the carbon dioxide hotspot, using the calculated amount of drones;
sampling the area, using the first drone, and updating the map with a new level of carbon dioxide.

18. The computer system of claim 17, wherein each drone of the amount of drones comprises an artificial photosynthesis module.

19. The computer system of claim 18, wherein the artificial photosynthesis module comprises a humidity unit, a photon generator, and a catalyst.

20. The computer system of claim 17, further comprising:
calculating the new level of carbon dioxide are above the threshold;
creating a new plan; and
diffusing the carbon dioxide using a calculated number of drones.
